(19) United States
(12) Patent Application Publication
(10) Pub. No.: US 2023/0222593 A1
(43) Pub. Date: Jul. 13, 2023

(54) METHOD AND SYSTEM FOR DETERMINING EQUITABLE BENEFIT IN DIGITAL PRODUCTS AND SERVICES

(71) Applicant: Akili Interactive Labs, Inc., Boston, MA (US)

(72) Inventors: Andrew C. Heusser, Newburyport, MA (US); Titiimaea Alailima, Cambridge, MA (US); Carl Gottlieb, Fairfax, CA (US)

(21) Appl. No.: 18/095,450

(22) Filed: Jan. 10, 2023

Related U.S. Application Data
(60) Provisional application No. 63/297,942, filed on Jan. 10, 2022.

(51) Int. Cl. G06Q 40/06

(52) U.S. Cl. CPC G06Q 40/06

(57) ABSTRACT

Methods, devices, systems and non-transitory computer-readable media having instructions stored thereon to enable one or more said methods, devices and systems for quantifying a degree of equitable benefit for a digital product or service. Certain objects and advantages of the present disclosure include a method and system to enable developers of software as a medical device and digital health intervention products and services to understand, track and improve a measure of equitable benefit for such products and services across a patient population, including across one or more patient demographics.

### CROSS-REFERENCE TO RELATED APPLICATIONS

This application claims priority to U.S. Provisional Application 63/297,942, filed Jan. 10, 2022, and entitled “METHOD AND SYSTEM FOR DETERMINING EQUITABLE BENEFIT IN DIGITAL PRODUCTS AND SERVICES,” the entirety of which is incorporated herein at least by virtue of this reference.

### FIELD

The present disclosure relates to the field of digital health platforms and products; in particular, a method and system to enable stakeholders of a digital health intervention to determine and assess a measure of equitable benefit for the digital health intervention across a patient population.

### BACKGROUND

Software is an increasingly critical area of healthcare product development. An expanding area of healthcare product development is in the area of digital health interventions (i.e., interventions delivered via digital technologies such as smartphones, mobile computing devices, wearable electronic devices, and the like) to provide effective, cost-effective, safe, and scalable interventions to improve health and healthcare. Digital health interventions (DHI) and Software as a Medical Device (SaMD) can be used to promote healthy behaviors, improve outcomes in people with long term conditions such as cardiovascular disease, diabetes and mental health conditions and provide remote access to effective treatments; for example, computerized cognitive behavioral therapy for mental health and somatic problems. Software as a Medical Device (SaMD) is defined by the International Medical Device Regulators Forum (IMDRF) as “software intended to be used for one or more medical purposes that perform these purposes without being part of a hardware medical device.” DHIs are often complex interventions with multiple components, and many have multiple aims including enabling users to be better informed about their health, share experiences with others in similar positions, change perceptions and cognitions around health, assess and monitor specified health states or health behaviors, titrate medication, clarify health priorities and reach treatment decisions congruent with these, and improve communication between patients and health care professionals (HCP). Active components may include information, psychoeducation, personal stories, formal decision aids, behavior change support, interactions with HCP and other patients, self-assessment or monitoring tools (questionnaires, wearables, monitors, and effective theory-based psychological interventions developed for face-to-face delivery such as cognitive behavioral therapy or mindfulness training). Certain DHI and SaMD products may include software that is itself directly therapeutically active in treating and/or targeting one or more neurological circuits related to one or more neurological, psychological and/or somatic conditions, diseases, and/or disorders, rather than just being a component of overall treatment.

The wide-spread adoption of technology products, including health-related technology products such as DHI and SaMD products, often presents asymmetrical challenges for differing segments of user populations; for example, age, gender, race, and the like. Through applied effort, ingenuity, and innovation, Applicant has identified deficiencies in prior art solutions for determining equitable benefit within user populations for digital products and services and has developed a solution that is embodied by the present disclosure, which is described in detail below.

### SUMMARY

In order to provide a basic understanding of the invention, the following is a simplified summary of certain embodiments thereof. This summary is not extensive and is not intended to identify key/critical elements of the invention or to delineate the scope of the invention. Its sole purpose is to present embodiments of the invention in a simplified form as a prelude to the more detailed description that is further below.

Certain aspects of the present disclosure provide for a method for quantifying a degree of equitable benefit (sometimes abbreviated herein as “EB”) for a digital product or service (e.g., a digital health intervention (DHI) or software as a medical device (SaMD) product or service). Certain aspects of the method may comprise one or more steps or operations for determining (e.g., with at least one processor) a demographic breakdown for at least one medical condition; determining (e.g., with at least one processor communicably engaged with at least one remote server) a target demographic breakdown of a patient population for the digital product or service; configuring (e.g., with the at least one processor) one or more benefit submetrics for the digital product or service; collecting (e.g., with an application server communicably engaged with a plurality of patient devices and the at least one processor) a plurality of patient activity data for the digital product or service; aggregating (e.g., with the at least one processor) the patient activity data; calculating (e.g., with the at least one processor) the one or more benefit submetrics for the digital product or service according to the patient activity data; processing (e.g., with the at least one processor) the one or more benefit submetrics according to an equitable benefit framework to calculate an equitable benefit score for the digital product or service; and providing (e.g., with the at least one processor communicably engaged with a client device) the equitable benefit score to a reviewer user at an interface of a reviewer application. In accordance with certain aspects of the present disclosure, the digital product or service is configured as a diagnostic or therapeutic for the at least one medical condition; for example, ADHD.

Certain aspects of the present disclosure provide for a method for quantifying a degree of equitable benefit for a digital product or service (e.g., DHI or SaMD product or service). Certain aspects of the method may comprise one or more steps or operations for establishing (e.g., via a network communications interface) a data transfer interface between an application server and a plurality of end user devices associated with a plurality of end users comprising a patient population; receiving (e.g., with the application server via the data transfer interface) personal demographic data for each end user in the plurality of end users; providing (e.g., with the application server) a software application to the plurality of end user devices; receiving (e.g., with the application server via the data transfer interface) device activation data for the software application from one or more end user device in the plurality of end user devices; executing, at one or more timepoints, one or more instances of the software application at the one or more end user device in the plurality of end user devices; receiving at the one or more timepoints (e.g., with the application server via the data transfer interface) a plurality of user activity data from the plurality of end user devices; communicating (e.g., with the application server) the personal demographic data for each end user in the plurality of end users and the plurality of user activity data from the plurality of end user devices to an analytics server via an application programming interface; segmenting (e.g., with the analytics server) the plurality of user activity data for each end user in the plurality of end users according to the personal demographic data for each end user; analyzing (e.g., with the analytics server) the plurality of user activity data according to an equitable benefit framework; generating (e.g., with the analytics server) an equitable benefit score for the software application; communicating (e.g., with the analytics server) the equitable benefit score for the software application to a client device communicably engaged with the analytics server; and displaying (e.g., with the client device) the equitable benefit score for the software application to at least one business user. wherein the equitable benefit framework comprises calculating one or more equitable benefit submetrics for one or more subgroups of the patient population. In certain embodiments, the plurality of user activity data comprises session data and one or more user-generated inputs from the one or more instances of the software application. In certain embodiments, the one or more equitable benefit submetrics comprise one or more of a user access metric, a product usage metric, and/or a user benefit metric for each end user in the plurality of end users. In certain embodiments, the equitable benefit score comprises comparing one or more calculated equitable benefit submetrics for the one or more subgroups of the patient population to one or more targeted distribution metrics for the patient population.

In accordance with certain aspects of the present disclosure, the method may be configured wherein calculating the device activation metric comprises one or more steps or operations for calculating (e.g., with the analytics server communicably engaged with at least one database) a ratio of delivered device activation codes to activated device activation codes stored in the at least one database. The method may be further configured wherein calculating the user compliance metric comprises comparing (e.g., with the analytics server communicably engaged with at least one database) an average number of sessions of the software application executed by each end user in the plurality of end users within a specified time period to a target number of sessions of the software application for the specified time period. The method may be further configured wherein calculating the user retention metric comprises comparing (e.g., with the analytics server communicably engaged with at least one database) a number of active sessions of the software application for each end user in the plurality of end users within a specified time period to a target number of active sessions of the software application for the specified time period. The method may be further configured wherein calculating the user benefit metric comprises tracking (e.g., with the analytics server communicably engaged with at least one database) at least one globally unique identifier for each end user in the plurality of end users. The at least one globally unique identifier may be embedded in at least one uniform resource locator associated with the software application (e.g., a webpage, download link, user signup workflow, etc.). In accordance with certain embodiments, the method may be configured wherein receiving the personal demographic data comprises receiving (e.g., with the application server via the data transfer interface) a plurality of user-generated responses via at least one end user workflow for the software application. The method may further comprise one or more steps or operations for establishing (e.g., with the analytics server or the application server) a data transfer interface with at least one electronic health records server, wherein the personal demographic data for each end user in the plurality of end users is received from the at least one electronic health records server. The method may further comprise one or more steps or operations for imputing (e.g., with the analytics server) one or more personal demographic datapoint for one or more end user in the plurality of end users according to one or more demographic statistics for the one or more end user in the plurality of end users. The method may further comprise one or more steps or operations for generating (e.g., with the analytics server) one or more recommendations for improving the equitable benefit score for the software application, wherein the one or more recommendations comprise recommendations for modifying one or more computerized stimuli or interactions for the software application for the one or more subgroups of the patient population.

Further aspects of the present disclosure provide for a system comprising an application server comprising a software application hosted thereon, the software application comprising a diagnostic or therapeutic application for at least one medical condition associated with a patient population; a plurality of end user computing devices communicably engaged with the application server via a network communications interface, the plurality of end user computing devices being associated with a plurality of end users comprising the patient population; an analytics server communicably engaged with the application server via at least one application programming interface; and a business user (e.g., a reviewer user) computing device communicably engaged with the analytics server. In accordance with certain aspects of the present disclosure, the application server is configured to execute one or more operations comprising: receiving personal demographic data for each end user in the plurality of end users from the plurality of end user computing devices; providing an instance of the software application to the plurality of end user computing devices; receiving device activation data for the software application from one or more end user device in the plurality of end user computing devices; receiving, at one or more timepoints, a plurality of user activity data from the plurality of end user computing devices, wherein the plurality of user activity data comprises session data and one or more user-generated inputs from one or more sessions of the software application, and communicating the personal demographic data and the plurality of user activity data to the analytics server via the at least one application programming interface. In accordance with certain aspects of the present disclosure, the analytics server is configured to execute one or more operations comprising: segmenting the plurality of user activity data for each end user in the plurality of end users according to the personal demographic data; analyzing the plurality of user activity data according to an equitable benefit framework, wherein the equitable benefit framework comprises calculating one or more equitable benefit submetrics for one or more subgroups of the patient population, wherein the one or more equitable benefit submetrics comprise one or more of a device activation metric, a user compliance metric, a user retention metric, and a user benefit metric for each end user in the plurality of end users; generating an equitable benefit score for the software application, wherein the equitable benefit score comprises comparing one or more calculated equitable benefit submetrics for the one or more subgroups of the patient population to one or more targeted distribution metrics for the patient population; and communicating the equitable benefit score for the software application to the business user computing device. In certain embodiments, the business user computing device is configured to display the equitable benefit score for the software application to at least one business user.

In accordance with certain embodiments of the system, the software application comprises one or more computerized stimuli or interactions configured to prompt one or more time varying responses from each end user in the plurality of end users via the plurality of end user computing devices, wherein the one or more computerized stimuli or interactions are associated with at least one computerized task. The analytics server may be further configured to execute one or more operations for generating one or more recommendations for improving the equitable benefit score for the software application, wherein the one or more recommendations comprise recommendations for modifying the one or more computerized stimuli or interactions for at least one end user in the plurality of end users. The application server may be further configured to execute one or more operations for modifying the one or more computerized stimuli or interactions for the at least one end user in the plurality of end users according to the one or more recommendations for improving the equitable benefit score for the software application. In certain embodiments, the business user computing device is configured to display the one or more recommendations for improving the equitable benefit score for the software application to the at least one business user. The analytics server is further configured to execute one or more operations for determining a measure of change in the equitable benefit score for the software application in response to modifying the one or more computerized stimuli or interactions for the at least one end user in the plurality of end users.

Still further aspects of the present disclosure provide for a non-transitory computer-readable medium having processor-executable instructions stored thereon that, when executed, command at least one processor to perform one or more operations, the one or more operations comprising: establishing a data transfer interface between an application server and a plurality of end user devices associated with a plurality of end users comprising a patient population; receiving personal demographic data for each end user in the plurality of end users; providing a software application to the plurality of end user devices, wherein the software application comprises a diagnostic or therapeutic application for at least one medical condition associated with the patient population; receiving device activation data for the software application from one or more end user device in the plurality of end user devices; receiving a plurality of user activity data from the plurality of end user devices, wherein the plurality of user activity data comprises session data and one or more user-generated inputs from one or more instances of the software application; segmenting the plurality of user activity data for each end user in the plurality of end users according to the personal demographic data for each end user; analyzing the plurality of user activity data according to an equitable benefit framework; generating an equitable benefit score for the software application; and communicating the equitable benefit score for the software application to a client device. In accordance with said aspects of the present disclosure, the equitable benefit framework comprises calculating one or more equitable benefit submetrics for one or more subgroups of the patient population. The one or more equitable benefit submetrics may comprise one or more of a device activation metric, a user compliance metric, a user retention metric, and a user benefit metric for each end user in the plurality of end users. The equitable benefit score may comprise comparing one or more calculated equitable benefit submetrics for the one or more subgroups of the patient population to one or more targeted distribution metrics for the patient population.

The foregoing has outlined rather broadly the more pertinent and important features of the present invention so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be recognized by those skilled in the art that such equivalent structures do not depart from the spirit and scope of the invention.

### BRIEF DESCRIPTION OF DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 is an architecture diagram of a system for determining equitable benefit for digital products and services, in accordance with certain aspects of the present disclosure;

FIG. 2 is a functional block diagram of a system for determining equitable benefit for digital products and services, in accordance with certain aspects of the present disclosure;

FIG. 3 is a functional block diagram of a system for determining equitable benefit for digital products and services, in accordance with certain aspects of the present disclosure;

FIG. 4 is a functional block diagram of a system for determining equitable benefit for digital products and services, in accordance with certain aspects of the present disclosure;

FIG. 5 is a process flow diagram of a routine for configuring an equitable benefit framework for a digital product or service, in accordance with certain aspects of the present disclosure;

FIG. 6 is a process flow diagram of a routine for collecting submetric data across a treatment cycle for a digital product or service, in accordance with certain aspects of the present disclosure;

FIG. 7 is a process flow diagram of a routine for calculating an equitable benefit score for a digital product or service, in accordance with certain aspects of the present disclosure;

FIG. 8 is a process flow diagram of a method for calculating an equitable benefit score for a digital product or service, in accordance with certain aspects of the present disclosure; and

FIG. 9 is a process flow diagram of a method for calculating an equitable benefit score for a digital product or service, in accordance with certain aspects of the present disclosure; and

FIG. 10 is a functional block diagram of an exemplary computing system through which certain aspects of the present disclosure may be implemented.

### DETAILED DESCRIPTION

It should be appreciated that all combinations of the concepts discussed in greater detail below (provided such concepts are not mutually inconsistent) are contemplated as being part of the inventive subject matter disclosed herein. It also should be appreciated that terminology explicitly employed herein that also may appear in any disclosure incorporated by reference should be accorded a meaning most consistent with the particular concepts disclosed herein.

Following below are more detailed descriptions of various concepts related to, and embodiments of, inventive methods, devices, systems and non-transitory computer-readable media having instructions stored thereon to enable one or more said methods, devices and systems for quantifying a degree of equitable benefit for a digital product or service, comprising determining a demographic breakdown for at least one medical condition; determining a target demographic breakdown of a patient population for the digital product or service, wherein the digital product or service is configured as a diagnostic or therapeutic for the at least one medical condition; configuring one or more benefit submetrics for the digital product or service; collecting a plurality of patient activity data for the digital product or service; aggregating the patient activity data; calculating the one or more benefit submetrics for the digital product or service according to the patient activity data; processing the one or more benefit submetrics according to an equitable benefit framework to calculate an equitable benefit score for the digital product or service; and providing the equitable benefit score to a reviewer user at an interface of a reviewer application.

It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes. The present disclosure should in no way be limited to the exemplary implementation and techniques illustrated in the drawings and described below.

Where a range of values is provided, it is understood that each intervening value, to the tenth of the unit of the lower limit unless the context clearly dictates otherwise, between the upper and lower limit of that range and any other stated or intervening value in that stated range is encompassed by the invention. The upper and lower limits of these smaller ranges may independently be included in the smaller ranges, and are also encompassed by the invention, subject to any specifically excluded limit in a stated range. Where a stated range includes one or both of the endpoint limits, ranges excluding either or both of those included endpoints are also included in the scope of the invention.

As used herein, “exemplary” means serving as an example or illustration and does not necessarily denote ideal or best.

As used herein, the term “includes” means includes but is not limited to, the term “including” means including but not limited to. The term “based on” means based at least in part on.

As used herein, the term “stimulus” refers to a sensory event configured to evoke a specified functional response from an individual. The degree and type of response can be quantified based on the individual's interactions with a measuring component (including using sensor devices or other measuring components).

As used in certain examples herein, the term “user activity data” refers to data collected from measures of an interaction of a user with a software program, product and/or platform.

As used herein, the term “computerized stimuli or interaction” or “CSI” refers to a computerized element that is presented to a user to facilitate the user's interaction with a stimulus or other interaction. As non-limiting examples, the computing device can be configured to present auditory stimulus (presented, e.g., as an auditory computerized adjustable element or an element of a computerized auditory task) or initiate other auditory-based interaction with the user, and/or to present vibrational stimuli (presented, e.g., as a vibrational computerized adjustable element or an element of a computerized vibrational task) or initiate other vibrational-based interaction with the user, and/or to present tactile stimuli (presented, e.g., as a tactile computerized adjustable element or an element of a computerized tactile task) or initiate other tactile-based interaction with the user, and/or to present visual stimuli or initiate other visual-based interaction with the user.

In an example where the computing device is configured to present visual CSI, the CSI can be rendered as at least one user interface to be presented to a user. In some examples, the at least one user interface is configured for measuring responses as the user interacts with a CSI computerized element rendered at the at least one user interface. In a non-limiting example, the user interface can be configured such that the CSI computerized element(s) are active, and may require at least one response from a user, such that the user interface is configured to measure data indicative of the type or degree of interaction of the user with the platform product. In another example, the user interface can be configured such that the CSI computerized element(s) are passive and are presented to the user using the at least one user interface but may not require a response from the user. In this example, the at least one user interface can be configured to exclude the recorded response of an interaction of the user, to apply a weighting factor to the data indicative of the response (e.g., to weight the response to lower or higher values), or to measure data indicative of the response of the user with the platform product as a measure of a misdirected response of the user (e.g., to issue a notification or other feedback to the user of the misdirected response).

As used in certain examples herein, the term “user” encompasses one or more of an end user and/or test user of a software program, product and/or platform and may further include: a patient being engaged with a software program, product or platform for a targeted medical or personal wellness purpose; a participant in a clinical trial, study or evaluation of a software program, product or platform; a user being engaged with a software program, product or platform for the purpose of evaluating or developing one or more technical, clinical, and/or functional aspects of a digital health intervention and/or a software as a medical device program, product or platform.

As used herein the terms “digital health intervention (DHI)” and “software as a medical device (SaMD)” may be used interchangeably and encompass any software program, product, or platform, including any software/hardware combination, being designed and/or utilized for any general or targeted medical or personal wellness purpose, including but not limited to the treatment, diagnosis, management, prevention, cure, or generation or provision of clinical, health, and/or wellness insights or recommendations to one or more users for one or more medical, health or personal wellness purpose; including any software program, product, or platform, including any software/hardware combination, being designed and/or utilized to promote healthy behaviors, improve outcomes in people with long term conditions such as cardiovascular disease, diabetes and mental health conditions and provide remote access to effective treatments; for example, computerized cognitive behavioral therapy for mental health and somatic problems; and may further encompass one or more software program, product or platform, including any product(s), program(s) and/or platform(s) that incorporate any combination of hardware and software, that is/are directly therapeutically active in treating and/or targeting one or more neurological circuits related to one or more neurological, psychological and/or somatic conditions, diseases, and/or disorders, rather than just being a component of overall treatment.

According to the principles herein, the term “session” refers to a discrete time period, with a clear start and finish, during which a user interacts with a software program, product, or platform to receive assessment or treatment therefrom.

All definitions, as defined and used herein, should be understood to control over dictionary definitions, definitions in documents incorporated by reference, and/or ordinary meanings of the defined terms. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

Certain objects and advantages of the present disclosure include a method and system to enable developers of SaMD and DHI products and services to understand, track and improve a measure of equitable benefit for such products and services across a patient population, including across one or more patient demographics.

Certain objects and advantages of the present disclosure include a method and system for targeting and increasing the size of end user populations for SaMD and DHI products and services.

In accordance with various aspects of the present disclosure, a digital product or service may comprise a DHI and/or SaMD platform, product, application, support tool and the like. In accordance with various aspects of the present disclosure, certain objects and advantages of a method and system for quantifying equitable benefit in digital products and services are to enable developers of said services to assess and evaluate product development and marketing efforts of digital products and services to ensure accessibility, use and benefit to patients and caregivers regardless of demographic background. In accordance with certain aspects of the present disclosure, a method and system for quantifying equitable benefit in digital products and services is configured to quantify measures of accessibility, use and benefit into an equitable benefit score, as described in more detail herein. In accordance with certain aspects of the present disclosure, an equitable benefit score comprises a quantitative summary of how well the demographics of a patient population for a digital product or service align with the demographics of the broader patient population for a medical condition (e.g., disease or disorder) that is targeted by the digital product or service; for example, pediatric ADHD.

Certain exemplary embodiments of the present disclosure may be configured as a processor-implemented system, method or apparatus that includes a display component, an input device, and the at least one processing unit. The at least one processing unit can be programmed to render at least one user interface, for display at the display component, to present the computerized stimuli or interaction (CSI) or other interactive elements to the user for interaction. In other examples, the at least one processing unit can be programmed to cause an actuating component of the platform product to effect auditory, tactile, or vibrational computerized elements (including CSIs) to effect the stimulus or other interaction with the user. Non-limiting examples of an input device include a touch-screen, or other pressure-sensitive or touch-sensitive surface, a motion sensor, a position sensor, a pressure sensor, and/or an image capture device (such as but not limited to a camera). The analysis of an end user's performance may include using the computing device to compute percent accuracy, number of hits and/or misses during a session or from a previously completed session. Other indicia that can be used to compute performance measures is the amount time the individual takes to respond after the presentation of a task (e.g., as a targeting stimulus). Other indicia can include, but are not limited to, reaction time, response variance, number of correct hits, omission errors, false alarms, learning rate, spatial deviance, subjective ratings, and/or performance threshold, etc. In a non-limiting example, the end user's performance can be further analyzed to compare the effects of two different types of tasks on the user's performances, where these tasks present different types of interferences (e.g., a distraction or an interrupter). The computing device is configured to present the different types of interference as CSIs or other interactive elements that divert the user's attention from a primary task. For a distraction, the computing device is configured to instruct the individual to provide a primary response to the primary task and not to provide a response (i.e., to ignore the distraction). For an interrupter, the computing device is configured to instruct the individual to provide a response as a secondary task, and the computing device is configured to obtain data indicative of the user's secondary response to the interrupter within a short time frame (including at substantially the same time) as the user's response to the primary task (where the response is collected using at least one input device). The computing device is configured to compute measures of one or more of a user's performance at the primary task without an interference, performance with the interference being a distraction, and performance with the interference being an interruption. The user's performance metrics can be computed based on these measures. For example, the user's performance can be computed as a cost (performance change) for each type of interference (e.g., distraction cost and interrupter/multi-tasking cost). The user's performance level on the tasks can be analyzed and reported as feedback, including either as feedback to the cognitive platform for use to adjust the difficulty level of the tasks, and/or as feedback to the individual concerning the user's status or progression.

In an exemplary embodiment, the system, method and apparatus of the present disclosure may include a computing device that is configured to present to a user a cognitive software application based on interference processing. In an example system, method and apparatus that implements interference processing, at least one processing unit is programmed to render at least one graphical user interface or cause an actuating component to generate an auditory, tactile, or vibrational signal, to present first CSIs as a first task that requires a first type of response from the end user. The example system, method and apparatus may also be configured to cause the at least one processing unit to render at least one second graphical user interface or cause the actuating component to generate an auditory, tactile, or vibrational signal, to present second CSIs as a first interference with the first task, requiring a second type of response from the user to the first task in the presence of the first interference. In a non-limiting example, the second type of response can include the first type of response to the first task and a secondary response to the first interference. In another non-limiting example, the second type of response may not include, i.e., may be different from, the first type of response. The at least one processing unit may also be programmed to receive data indicative of the first type of response and the second type of response based on the user interaction with the platform product, such as but not limited to by rendering the at least one graphical user interface to receive the data.

As shown in Table 1 below, an equitable benefit analytical framework may incorporate demographic data for a patient population associated with a medical condition (e.g., pediatric ADHD).

TABLE 1
Exemplary Demographic Dataset for
Pediatric ADHD Patient Population

| Patient Characteristic | | Percent with ADHD |
|---|---|---|
| Age Group | — | — |
| | 5-17 years | 10.8 |
| | 5-9 years | 7.4 |
| | 10-17 years | 12.9 |
| Sex | — | — |
| | Male | 14.6 |
| | Female | 6.9 |
| Race | — | |
| | White only | 10.8 |
| | Black or African | 13.2 |
| | American only | |
| | American Indian or | 6.4 |
| | Alaska Native only | |
| | Asian only | 3.1 |
| | 2 or more races | 14.5 |
| Hispanic origin | — | — |
| and race | Hispanic or Latino | 7.3 |
| | Not Hispanic or Latino | 11.9 |
| | White only | 12.3 |
| | Black or African | 13.4 |
| | American only | |
| Percent of | — | — |
| poverty level | Below 100% | 13.5 |
| | 100%-199% | 11.7 |
| | 200%-399% | 9.7 |
| | 400% or more | 9.6 |
| Health | — | — |
| insurance status | Insured | 11.0 |
| at time of | Private | 9.2 |
| interview | Medicaid | 13.8 |
| | Uninsured | 6.7 |

To go from prevalence rates to a count of the number of children with ADHD in a specific geography (broken down by demographic), an equitable benefit framework may be configured to multiply the prevalence rates with the total number of children in the specific geography (also broken down by demographic) and then normalize (sums to 1) to create a probability distribution. In accordance with certain aspects of the present disclosure, an equitable benefit score is quantified as a number between 0 and 1; where 1 represents a target patient population that is perfectly aligned with the broader patient population for the medical condition. In accordance with certain aspects of the present disclosure, the equitable benefit score may be measured for different demographics within the patient population as well as for different subgroups of the patient population at key points along the patient journey (i.e., milestones during a treatment cycle) for the digital product or service. The demographic data from Table 1 may be obtained via one or more third-party database (including at least one electronic health records (HER) database) and/or may be self-reported by the patient population for the digital product or services and/or may be imputed according to one or more methods. For example, census data may be utilized to impute race, ethnicity and income based on zip code. Zip code-based imputation is a way to fill in missing data if an end user decides not to provide such data or the data is missing for other reasons. The basis for zip code-based imputation is that race, ethnicity and income tend to systematically vary from zip code to zip code, and so if a patient does not provide this information, the system may impute a “best guess” based on where the patient lives. Zip code-based imputation works by using the patient's zip code to look up demographic statistics from census data. Then, a demographic “label” (e.g., Income >100k or Income <=100k as a simple example) is sampled and fills in the missing value. This process is repeated a number of times to build up a distribution of equitable benefit scores that represents a degree of uncertainty for the final score due to the imputation procedure.

In accordance with certain aspects of the present disclosure, an equitable benefit framework may comprise one or more submetrics. In accordance with certain embodiments, the submetrics may comprise a user access submetric, a product usage submetric and a user benefit submetric. In certain embodiments, the product usage submetric may further comprise a user compliance submetric and a user retention submetric. The submetrics may by calculated for one or more subgroups of patients that have achieved certain milestones in a treatment cycle of a SaMD or DHI product. The primary motivation for multiple metrics is to assess whether the digital product or service is equitable not only to those who begin treatment, but also for the subset of patients who use the digital product or service as it was designed to be used; and importantly, those who ultimately benefit from the treatment. In accordance with certain aspects of the present disclosure, the submetrics are filters that select subgroups of patients who have hit certain criteria in the treatment cycle. An access submetric may analyze one or more data points to measure or quantify a number or percentage of users who have started treatment on the digital product or service. In accordance with certain aspects of the present disclosure, the compliance submetric may analyze one or more parameters for determining compliance with the treatment according to the equitable benefit framework. The retention submetric may analyze one or more parameters for determining adherence to the treatment during a specific timeframe. In accordance with certain aspects of the present disclosure, the benefit submetric may analyze one or more data points to measure or quantify a number or percentage of users who have received a benefit (e.g., improvement in ability and/or reduction in symptoms) from the digital product or services. For example, a benefit metric may comprise a measure of patients who respond “very likely” to recommend the digital products or services to a friend or colleague in a survey and/or a measure of efficacy for the digital products or services based on one or more user performance data.

In accordance with certain aspects of the present disclosure, the equitable benefit framework may be configured to calculate a “Total EB Score” comprising demographic breakdowns of a patient population to a target demographic breakdown(s) for one or more submetrics, which are then aggregated and averaged to obtain an overall EB score. In accordance with certain aspects of the present disclosure, the equitable benefit framework may be configured to compute the distance between patient demographic distributions (e.g., gender, race, ethnicity and income) and their respective target distributions (e.g., American children under 18 with ADHD) for each submetric (e.g., access, usage, compliance, retention, benefit, etc.). In accordance with certain aspects of the present disclosure, the equitable benefit framework may comprise a distance function to compare these distributions. In accordance with certain aspects of the present disclosure, the distance function may comprise two primary advantages to a typical distance function for probability distributions. First, the distance function may assign higher importance to smaller groups (e.g., a difference between the total patient population versus a target in Black/African American or Native American patients may carry more weight than White patients). Second, non-linear distances from 0 to 1 (e.g., improvement in EB Score is increasingly more difficult as the score approaches 1). In accordance with certain aspects of the present disclosure, the equitable benefit framework may be configured to calculate a total EB Score by aggregating and averaging the distances for all demographic variables and submetrics. In accordance with certain embodiments of the equitable benefit framework, all demographic variables may be weighted equally and the submetrics may be weighted, for example, as follows: Access (⅓), Compliance (⅙), Retention (⅙), Benefit (⅓).

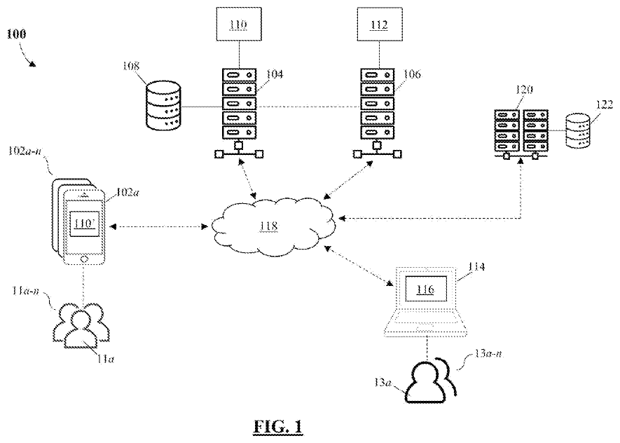

Turning now descriptively to the drawings, in which similar reference characters denote similar elements throughout the several views, FIG. 1 depicts an architecture diagram of a system 100 for determining a measure of equitable benefit for digital products and services. A digital product or service may comprise a SaMD and/or a DHI product, platform and/or application. In accordance with certain aspects of the present disclosure, system 100 may be configured to measure a degree of equitable benefit for one or more of an end user population, a subgroup of end users and one or more individual end users of the digital products and services. A measure of equitable benefit may include an equitable benefit score. The equitable benefit score may represent a degree to which an intended or targeted benefit of the digital products and services compares to a measured or actual level of benefit in the end user population, the subgroup of end users and/or the one or more individual end users. Additionally, or alternatively, the equitable benefit score may represent a degree to which an end user population for the digital products and services is reflective of one or more statistical characteristics (e.g., demographic characteristics) of a patient population for at least one medical condition. In certain embodiments, the at least one medical condition comprises a cognitive condition or disorder, such as ADHD. In accordance with certain aspects of the present disclosure, system 100 may be configured to make one or more real-time or ad hoc modifications to one or more application logic or CSIs of the digital product or service in response to the equitable benefit score.

In accordance with certain aspects of the present disclosure, system 100 may comprise an application server 104 communicably engaged with an analytics server 106 via one or more data transfer interface, such as an application programming interface, software development kit, and/or one or more other data transfer protocols via a network communications interface (e.g., network interface 118). Application server 104 may host a software application 110 thereon. Software application 110 may comprise a SaMD or DHI application associated with at least one medical condition (e.g., ADHD). Application server 104 may provide an instance 110′ of software application 110 to one or more client device 102a in a plurality of client devices 102a-n. In certain embodiments, client device 102a may comprise a portable computing device, such as a smartphone or tablet computer, comprising one or more sensors configured to measure one or more inputs from end user 11a both temporally and spatially. The one or more sensors may comprise one or more of a gyroscope, an accelerometer, an e-compass, a motion sensor, a position sensor, a pressure sensor, an optical sensor, a video camera, an auditory sensor, a vibrational sensor, and the like. Instance 110′ may comprise a native instance of software application 110 being installed on client device 102a and/or may comprise a web-based instance being delivered via a web browser of client device 102a and/or a hybrid thereof. Plurality of client devices 102a-n may be associated with a plurality of end users 11a-n comprising a user population for software application 110. Plurality of end users 11a-n may comprise a plurality of individual who have a cognitive disease, disorder or condition that may be diagnosed, treated and/or monitored via one or more session interactions with software application 110. In accordance with certain aspects of the present disclosure, system 100 is configured to analyze a plurality of user-generated data collected during one or more sessions interactions with software application 110 to determine a degree to which an individual end user 11a and/or a subgroup or a total number of end users in plurality of end users 11a-n derives an expected or target outcome or benefit from the one or more session interactions with software application 110. In accordance with certain aspects of the present disclosure, system 100 is configured to analyze a degree to which software application 110 is effective in delivering an equitable benefit to one or more subgroups of end users in plurality of end users 11a-n when comparing one or more demographic characteristics of the one or more subgroups of plurality of end users 11a-n to one or more demographic characteristics of a total patient population for the cognitive disease, disorder or condition.

In accordance with certain aspects of the present disclosure, application server 104 may be configured to provide instance 110′ of software application 110 to end user device 102a via network interface 118. In certain embodiments, application server 104 may be configured to generate an activation code for instance 110′ and communicate the activation code to end user device 102a via network interface 118. In certain embodiments, end user 11a may input the activation code via a new user workflow for software application 110. The new user workflow may comprise a graphical user interface including one or more prompts for end user 11a to input personal demographic data. End user device 102a may be configured to communicate the activation code and the personal demographic data for end user 11a to application server 104 via network interface 118. Application server 104 may store the activation code and the personal demographic data for end user 11a in application database 108. This process may be repeated, sequentially or concurrently, for each end user in the plurality of end users 11a-n via each end user device in the plurality of end user devices 102a-n.

Subsequent to activation of software application 110 and completion of the new user setup/workflow (if applicable), end user device 102a may be communicably engaged with application server 104 to render one or more sessions of instance 110′ of software application 110 to end user 11a at one or more time points. In certain embodiments, software application 110 may comprise one or more CSIs comprising one or more computerized tasks for the user to complete within a session of instance 110′. End user device 102a may receive one or more user-generated inputs (i.e., responses) from end user 11a in response to the one or more CSIs within the session of instance 110′ and may communicate data generated from the one or more user-generated inputs (e.g., user activity data) to application server 104 via communications interface 118. Application server 104 may be communicably engaged with application database 108 to store the session data from end user 11a in application database 108. This process may be repeated, sequentially or concurrently, for each end user in the plurality of end users 11a-n via each end user device in the plurality of end user devices 102a-n.

In accordance with certain aspects of the present disclosure, application server 104 may be communicably engaged with analytics server 106 via an application programming interface (or other data transfer protocol) to communicate one or more of the activation data, the personal demographic data, and the user-activity data from one or more sessions for plurality of end users 11a-n to analytics server 106. In certain embodiments, system 100 may comprise at least one third-party server 120 communicably engaged with application server 104 and/or analytics server 106. Third-party server 120 may be communicably engaged with at least one third-party database 122 to provide one or more demographic data for one or more patient population for at least one medical condition and/or one or more statistical or other data for the at least one medical condition. In certain embodiments, third-party server 120 is a public health system server. In certain embodiments, third-party server 120 is an electronic medical records server and third-party database 122 is an electronic medical records database comprising one or more medical records data for plurality of end users 11a-n. In accordance with certain aspects of the present disclosure, analytics server 106 may comprise an equitable benefit (EB) framework 112 configured to receive and process the data received from application server 104 and, optionally, the data received from third-party server 120 to calculate an equitable benefit score for plurality of end users 11a-n and/or one or more subgroup of the plurality of end users 11a-n and/or an individual end user in the plurality of end users 11a-n (e.g., end user 11a). System 100 may further comprise one or more reviewer device 114 communicably engaged with analytics server 106 and/or application server 104 via network interface 118. In certain embodiments, analytics server 106 may be configured to communicate the equitable benefit score to reviewer device 114. Reviewer device 114 may be configured to render the equitable benefit score to one or more reviewer users 13a-n via a reviewer interface 116. In certain embodiments, EB framework 112 may comprise one or more machine learning model and/or rules engine configured to generate one or more recommendations for improving the equitable benefit score based on the data received from application server 104 and, optionally, the data received from third-party server 120. In said embodiments, analytics server 106 may be configured to communicate the one or more recommendations to reviewer device 114. The one or more recommendations may be rendered at reviewer interface 116. In certain embodiments, reviewer interface 116 comprises a graphical user interface configured to receive one or more inputs from a reviewer user 13a. The one or more inputs from a reviewer user 13a may comprise one or more inputs for configuring one or more elements of EB framework 112 and/or modifying one or more CSIs or user prompts for software application 110.

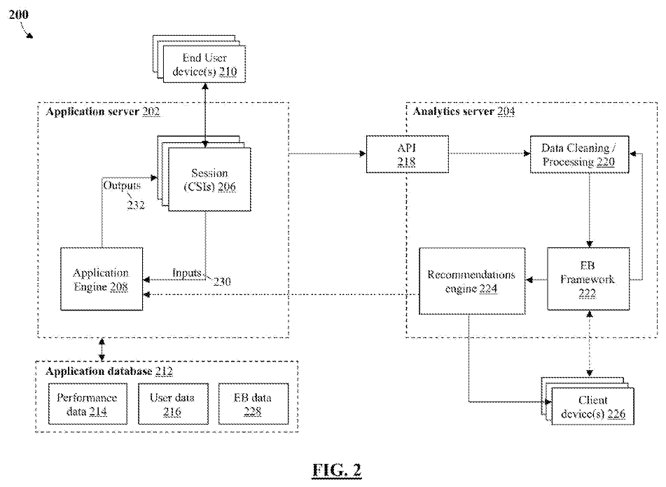

Referring now to FIG. 2, a functional block diagram of a system 200 for determining equitable benefit for digital products and services is shown. In accordance with certain aspects of the present disclosure, system 200 may comprise an embodiment of system 100 of FIG. 1. System 200 may comprise an application server 202 comprising a SaMD or DHI application or software product hosted thereon. In certain embodiments, the SaMD or DHI application or software product is operably configured for at least one intended medical or clinical use or purpose associated with at least one medical condition; including a diagnostic tool or therapeutic treatment. The at least one medical condition may comprise a cognitive disease, disorder, or condition; for example, ADHD. The SaMD or DHI application or software product may comprise one or more computerized stimuli or interactions (CSIs) configured to elicit a targeted stimulus-response pattern from an end user. The targeted stimulus-response pattern may comprise a clinically-validated stimulus-response framework associated with the at least one medical condition; for example, a selective stimulus management (SSM) framework. In certain embodiments, application server 202 is configured to instantiate one or more sessions 206 of the SaMD or DHI application or software product at one or more end user devices 210. Application server 202 comprises an application engine 208 configured to drive the presentation and configuration of one or more CSIs within the one or more sessions 206 of the SaMD or DHI application or software product. End user devices 210 are configured to receive a plurality of user-generated responses within the one or more sessions 206, including one or more time-varying response to the one or more CSIs, from one or more end users. The one or more end users may comprise an end user population for the SaMD or DHI application or software product.

In accordance with certain aspects of the present disclosure, the plurality of user-generated responses may be configured as one or more inputs 230 to application engine 208. Application engine 208 may be configured to process inputs 230 to drive one or more outputs 232 at the one or more sessions 206 of the SaMD or DHI application or software product. In certain embodiments, the one or more output may comprise one or more dynamic modifications of the CSIs (including a modification in difficulty level of at least one computerized task) and/or presentation or modification of one or more graphical elements of a graphical user interface of the SaMD or DHI application or software product. System 200 may further comprise an application database 212 communicably engaged with application server 202. Application server 202 may be configured to communicate inputs 230 to application database 212, which may be stored in application database as performance data 214. In certain embodiments, the one or more sessions 206 of the SaMD or DHI application or software product may comprise one or more user workflow or session prompts configured to elicit personal demographic data from the one or more users of end user devices 210. The personal demographic data may be stored in application database 212 as user data 216.

In accordance with certain aspects of the present disclosure, application server 202 may be communicably engaged with an analytics server 204 via an application programming interface (API) 218. Application server 202 may be configured to communicate inputs 230 received from end user devices 210 in real-time via API and/or may be configured to communicate performance data 214 and user data 216 according to one or more batch transfer protocols and/or ad hoc in response to one or more API calls received from analytics server 204. Analytics server 204 may, optionally, comprise at least one data processing and cleaning module 220 configured to receive, process and clean the data received from application server 202 via one or more computational or data processing frameworks. Certain data cleaning techniques suitable for use in various embodiments include, without limitation, imputation, capping, and flooring of the data. Data processing and cleaning module 220 may be dynamically configured according to one or more parameters configured to analyze one or more dependent variables associated with the intended medical or clinical use or purpose (e.g., efficacy) of the SaMD or DHI application or software product. Data processing and cleaning module 220 may be further configured to execute one or more data management operations configured to segment, classify, and/or process the data. In accordance with certain aspects of the present disclosure, data processed at the data processing and cleaning module 220 may be further processed according to an equitable benefit framework 222. Equitable benefit framework 222 may be configured to analyze one or more submetrics from the data in order to calculate an equitable benefit score for the SaMD or DHI application or software product. In certain embodiments, the one or more submetrics may be comprise one more user access submetric, one or more use or engagement submetric, and one or more user benefit submetric. The one or more submetrics may be calculated for one or more of an individual user, a subgroup of users in the user population, and/or the total user population. In certain embodiments, the user access submetric may comprise a device activation metric. In said embodiments, equitable benefit framework 222 may be configured to analyze device activation code data received from application server 202 to calculate a ratio of delivered device activation codes to activated device activation codes stored in application database 212 in order to derive the user access submetric. In certain embodiments, the one or more usage or engagement submetric may comprise a user compliance metric and/or a user retention metric. Equitable benefit framework 222 may be configured to calculate the user compliance metric by executing one or more operations for comparing an average number of sessions 206 executed by each end user in a plurality of end users within a specified time period to a target number of sessions 206 for the specified time period (e.g., 10 session/week average for four weeks). Equitable benefit framework 222 may be configured to calculate the user retention metric by executing one or more operations for comparing a number of active sessions 206 for each end user in the plurality of end users within a specified time period to a target number of active sessions 206 for the specified time period (e.g., 5 or more sessions within a 30-day period). Equitable benefit framework 222 may be configured to calculate the user benefit metric by executing one or more operations for evaluating a degree of user satisfaction for the SaMD or DHI application or software product and/or measuring a degree of efficacy for the SaMD or DHI application or software product. In accordance with certain embodiments, equitable benefit framework 222 may be configured to determine a measure of efficacy of the SaMD or DHI application or software product by analyzing performance data 214 to determine one or more actual stimulus-input patterns from performance data 214 for one or more end users and comparing the one or more actual stimulus-input patterns to one or more expected stimulus-input patterns for the SaMD or DHI application or software product. In accordance with certain embodiments, equitable benefit framework 222 may be configured to analyze one or more of the calculated submetrics to generate an equitable benefit score for the SaMD or DHI application or software product. Analytics server 204 may be configured to communicate the equitable benefit score to one or more client devices 226. In certain embodiments, one or more client devices 226 may be associated with one or more reviewer users.

In accordance with certain aspects of the present disclosure, equitable benefit framework 222 may be configured to provide the equitable benefit score, and/or one or more of the calculated submetrics, to a recommendations engine 224. Recommendations engine 224 may comprise one or more rules engine or machine learning model configured to analyze one or more of the performance data 214, user data 216, calculated submetric data and the equitable benefit score to generate one or more recommendations for improving the equitable benefit score. In certain embodiments, analytics server 204 may be configured to communicate the one or more recommendations for improving the equitable benefit score to one or more client devices 226. In certain embodiments, the one or more client devices 226 may comprise a reviewer application executing thereon comprising a graphical user interface configured to enable at least one review user to configure one or more modifications to the equitable benefit framework 222 and/or the SaMD or DHI application or software product. In certain embodiments, analytics server 204 may be configured to communicate one or more outputs from EB framework 222 (e.g., the equitable benefit score) and/or recommendations engine 224 (e.g., one or more recommendations) to application server 202. Application server 202 may be configured to store the equitable benefit score and/or the one or more recommendations as equitable benefit data 228 in the application database. In accordance with certain aspects of the present disclosure, application engine 208 may be configured to receive the equitable benefit data from analytics server 204 and process the data as an input 230. Application engine 208 may be configured to dynamically modify one or more CSIs (including a modification in difficulty level of at least one computerized task) and/or dynamically present, modify, or configure one or more graphical elements of the graphical user interface of the SaMD or DHI application or software product (e.g., as one or more outputs 232) according to the equitable benefit data. In accordance with certain aspects of the present disclosure, application server 202 is configured to present one or more subsequent sessions 206 of the SaMD or DHI application or software product to end user devices 210 and collect one or more subsequent sets of performance data 214 and/or user data 216. The one or more subsequent sets of performance data 214 and/or user data 216 may be communicated to analytics server 204 via API 218 and analyzed according to EB framework 222 to generate an updated equitable benefit score. Recommendations engine 224 may be configured to analyze the updated equitable benefit score and/or the one or more subsequent sets of performance data 214 and/or user data 216 to determine a degree of improvement for the equitable benefit score in response to one or more prior recommendations (e.g., modifications).

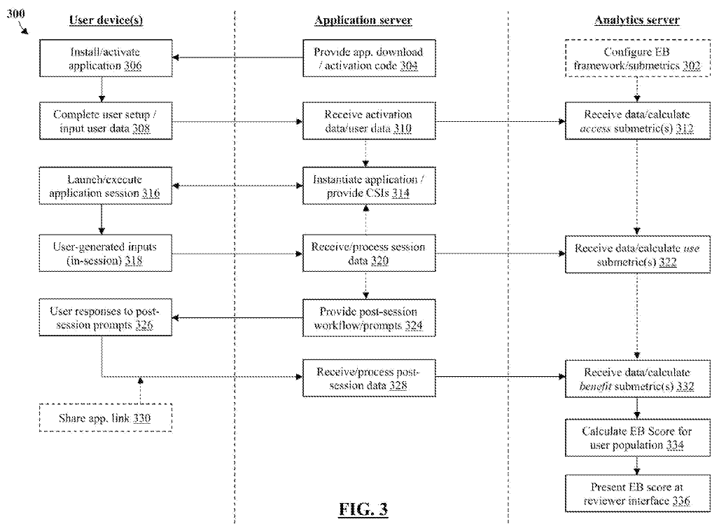

Referring now to FIG. 3, a functional block diagram of a process flow 300 of a system for determining equitable benefit for digital products and services is shown. In accordance with certain aspects of the present disclosure, the system for determining equitable benefit for digital products and services may comprise system 100, as shown in FIG. 1, and/or system 200, as shown in FIG. 2. Process flow 300 may comprise one or more process steps 302-336 for calculating an equitable benefit score for a user population of a SaMD or DHI application or software product and presenting the equitable benefit score to at least one reviewer user at a reviewer interface. The one or more process steps 302-336 may be executed across one or more communicably-engaged system components including, but not limited to, an application server, one or more user devices, and an analytics server. In accordance with certain aspects of the present disclosure, process flow 300 may comprise one or more operations for configuring an equitable benefit framework at the analytics server (Step 302). In certain embodiments, the equitable benefit framework may comprise one or more submetrics; optionally including a user access submetric, a product usage submetric, and a user benefit submetric. In accordance with certain aspects of the present disclosure, process flow 300 may comprise one or more operations for providing a download of a software application (e.g., the SaMD or DHI application) to one or more user devices (Step 304). Step 304 may comprise providing an activation code for the software application to the one or more user devices. The one or more user devices may be associated with one or more end users of the software application. The end users may perform one or more operations to install the software application at the one or more user devices and activate the software application (e.g., by entering the activation code at a graphical user interface of an instance of the software application) (Step 306). In certain embodiments, the end users may complete a new user setup/workflow within a graphical user interface of the software application (Step 308). The new user setup/workflow may comprise one or more fields/prompts configured to elicit one or more personal demographic data inputs from the end users. Process flow 300 may comprise one or more operations for communicating the user data (optionally including the activation code) from the one or more user devices to the application server and receiving the user data (optionally including the activation code) at the application server (Step 310). Process flow 300 may comprise one or more operations for communicating the user data (optionally including the activation code) from the application server to the analytics server and receiving the user data (optionally including the activation code) at the analytics server (Step 312). Step 312 may further comprise one or more operations for processing the user data according to the equitable benefit framework to calculate at least one user access submetric for one or more users.

In accordance with certain aspects of the present disclosure, process flow 300 may comprise one or more operations for launching/executing a session of the software application at the user device(s) (Step 316) and instantiating the session of the software application at the application server (Step 314). Step 314 may comprise one or more operations for providing one or more CSIs within the session of the software application to the user device(s). Process flow 300 may comprise one or more operations for receiving one or more user-generated inputs within the session of the software application via the user device(s) (e.g., in response to the one or more CSIs) (Step 318). Process flow 300 may comprise one or more operations for communicating data from the one or more user-generated inputs (e.g., session data) from the user devices to the application server and receiving/processing the session data at the application server (Step 320). Process flow 300 may comprise one or more operations for communicating the session data from the application server to the analytics server and receiving the session user data at the analytics server (Step 322). Step 322 may further comprise one or more operations for processing the session data according to the equitable benefit framework to calculate at least one product usage submetric for the one or more users. In certain embodiments, the product usage submetric may comprise a user compliance metric and/or a user adherence metric. In accordance with certain aspects of the present disclosure, process flow 300 may repeat one or more of steps 314-322 at one or more timepoints comprising one or more sessions of the software application. Process flow 300 may further comprise one or more operations for configuring and/or providing one or more post-session workflow and/or user prompts to the user devices (Step 324). Process flow 300 may further comprise one or more operations for receiving one or more user-generated response from the one or more end users via the user devices in response to the one or more post-session workflow and/or user prompts (Step 326). In certain embodiments, the one or more post-session workflow and/or user prompts may comprise presenting at least one hyperlink to the one or more end users for sharing a download or user signup page for the software application. In certain embodiments, Step 324 may comprise one or more operations for embedding a globally unique identifier for each end user in the one or more end users in the at least one hyperlink, such that the sharing activity of the at least one hyperlink may be tracked across each end user in the one or more end users. In said embodiments, process flow 300 may comprise one or more operations for sharing the at least one hyperlink from the one or more user devices (Step 330). Process flow 300 may comprise one or more operations for communicating post-session data from the user devices and receiving/processing the post-session data (optionally including the hyperlink sharing data) at the application server (Step 328). Process flow 300 may comprise one or more operations for communicating the post-session data from the application server to the analytics server and receiving the post-session data at the analytics server (Step 332). Step 332 may further comprise one or more operations for processing the post-session data according to the equitable benefit framework to calculate at least one user benefit submetric for the one or more users. In certain embodiments, calculating the at least one user benefit submetric comprises calculating a measure of efficacy for the software application for one or more end users. In certain embodiments, calculating the at least one user benefit submetric comprises calculating a measure of user satisfaction from the one or more end users. Process flow 300 may comprise one or more operations for processing one or more of the equitable benefit submetrics to calculate an equitable benefit score for the user population of a SaMD or DHI application or software product (Step 334). Process flow 300 may comprise one or more operations for presenting the equitable benefit score to at least one reviewer user at a reviewer interface (Step 336).

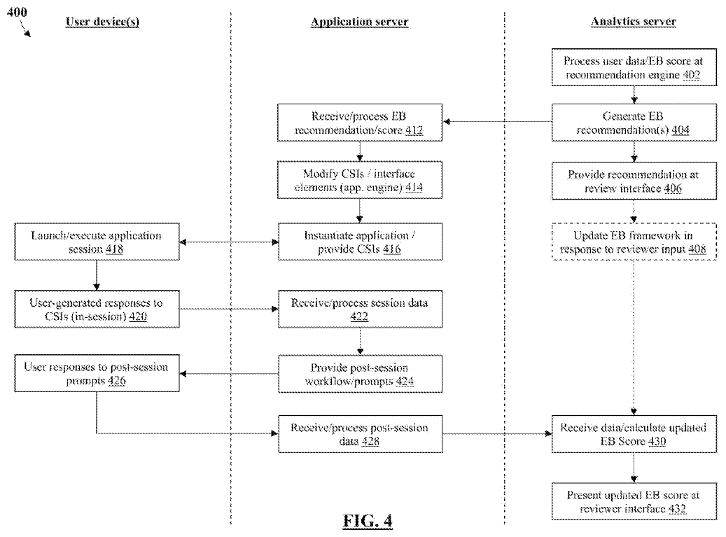

Referring now to FIG. 4, a functional block diagram of a process flow 400 of a system for determining equitable benefit for digital products and services is shown. In accordance with certain aspects of the present disclosure, the system for determining equitable benefit for digital products and services may comprise system 100, as shown in FIG. 1, and/or system 200, as shown in FIG. 2. Process flow 400 may be successive or sequential to one or more steps or operations of process flow 300, as shown in FIG. 3, and/or may comprise one or more sub-steps or sub-operations of process flow 300. Process flow 400 may comprise one or more process steps 402-432 for modifying one or more CSIs or graphical user interface elements of a SaMD or DHI application or software product in response to one or more generated equitable benefit recommendations and calculating an updated equitable benefit score in response to modifying the one or more CSIs or graphical user interface elements. The one or more process steps 402-432 may be executed across one or more communicably-engaged system components including, but not limited to, an application server, one or more user devices, and an analytics server. In accordance with certain aspects of the present disclosure, process flow 400 may comprise one or more operations for processing a plurality of user data (e.g., session data and/or post-session data from process flow 300) and/or a calculated equitable benefit score for the SaMD or DHI application or software product (e.g., as calculated pursuant to process flow 300) via a recommendation engine of the analytics server (Step 402). The recommendation engine may comprise one or more machine learning framework and/or rules engine configured to process the plurality of user data and/or the calculated equitable benefit score to generate one or more recommendations for improving the equitable benefit score for at least one or more users of the SaMD or DHI application or software product (Step 404). Process flow 400 may comprise one or more operations for providing the one or more recommendations for improving the equitable benefit score to at least one reviewer user at a reviewer interface (Step 406). In certain embodiments, process flow 400 may comprise one or more operations for updating the equitable benefit framework at the analytics server in response to one or more reviewer inputs received at the reviewer interface in response to the one or more recommendations for improving the equitable benefit score (Step 408).

In accordance with certain aspects of the present disclosure, process flow 400 may comprise one or more operations for communicating the equitable benefit score and/or the one or more recommendations for improving the equitable benefit score from the analytics server to the application server and receiving/processing the one or more recommendations and/or the equitable benefit score at the application server (Step 412). In certain embodiments, Step 412 may comprise processing the one or more recommendations and/or the equitable benefit score at an application engine of the application server. Process flow 400 may comprise one or more operations for modifying (either in real-time or for one or more subsequent sessions) one or more CSIs and/or graphical user interface elements of the software application (Step 414). In accordance with certain aspects of the present disclosure, process flow 400 may comprise one or more operations for launching/executing a subsequent session of the software application at the user device(s) (Step 418) and instantiating the subsequent session of the software application at the application server (Step 416). Step 414 may comprise one or more operations for providing one or more modified CSIs and/or modified graphical user interface elements configured at Step 414 at the subsequent session of the software application. Process flow 400 may comprise one or more operations for receiving one or more user-generated inputs within the subsequent session of the software application via the user device(s) (e.g., in response to the one or more CSIs) (Step 420). Process flow 400 may comprise one or more operations for communicating data from the one or more user-generated inputs (e.g., session data) from the user devices to the application server and receiving/processing the session data at the application server (Step 422). Process flow 400 may further comprise one or more operations for configuring one or more subsequent post-session workflow and/or user prompts and providing the one or more subsequent post-session workflow and/or user prompts to the user devices (Step 424). Process flow 400 may further comprise one or more operations for receiving one or more user-generated response from the one or more end users via the user devices in response to the one or more post-session workflow and/or user prompts (Step 426). Process flow 400 may comprise one or more operations for communicating post-session data from the user devices to the application server and receiving/processing the post-session data at the application server (Step 428). Process flow 400 may comprise one or more operations for communicating the session data and the post-session data from the application server to the analytics server and receiving/processing the session data and the post-session data at the analytics server (Step 430). Step 430 may further comprise one or more operations for processing the session data and the post-session data according to the equitable benefit framework to calculate an updated equitable benefit score for the user population of a SaMD or DHI application or software product. The updated equitable benefit score may comprise a measure of one or more improvements to one or more prior equitable benefit score for the user population in response to implementation of one or more equitable benefit recommendations at the application server. Process flow 400 may comprise one or more operations for presenting the updated equitable benefit score to at least one reviewer user at the reviewer interface (Step 432).

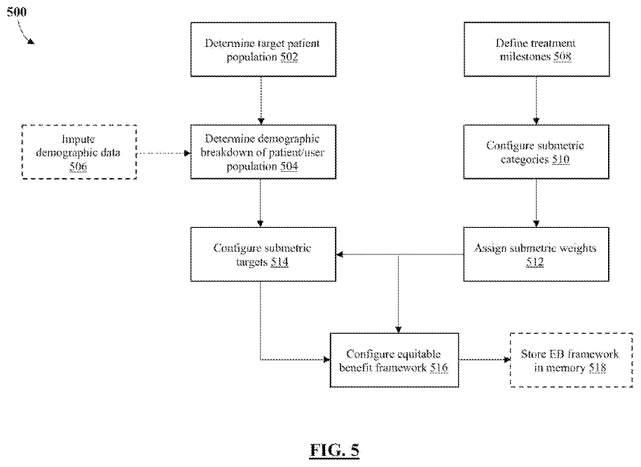

Referring now to FIG. 5, a process flow diagram of a routine 500 for configuring an equitable benefit framework for a digital product or service is shown. In accordance with certain aspects of the present disclosure, routine 500 may be embodied within one or more system routine of system 100, as shown in FIG. 1, and/or system 200, as shown in FIG. 2. The digital product or service may comprise a DHI or an SaMD platform and/or product and may be configured as a digital therapeutic or diagnostic tool for treating or diagnosing a targeted medical condition (e.g., pediatric ADHD). In accordance with certain aspects of the present disclosure, routine 500 may comprise one or more steps or operations for determining a patient population for a targeted medical condition (Step 502); for example, the patient population for pediatric ADHD in America. Routine 500 may comprise one or more steps or operations for determining a demographic breakdown of a patient population for the digital product or service (i.e., users) (Step 504). In accordance with certain aspects of the present disclosure, routine 500 may comprise one or more steps or operations for imputing demographic data for the patient population for the digital product or service according to one or more imputation methods (e.g., zip code-based imputation) (Step 506). In accordance with certain aspects of the present disclosure, routine 500 may comprise one or more steps or operations for defining one or more treatment milestones for the digital product or service (Step 508). In accordance with certain aspects of the present disclosure, the digital product or service may be configured as a digital therapy or diagnostic (e.g., DHI/SaMD) for treating or diagnosing the targeted medical condition. In accordance with certain aspects of the present disclosure, routine 500 may comprise one or more steps or operations for configuring one or more submetric categories for a treatment cycle of the digital product or service (Step 510). In accordance with certain aspects of the present disclosure, routine 500 may comprise one or more steps or operations for assigning weights to each of the submetric categories (Step 512). In accordance with certain aspects of the present disclosure, routine 500 may comprise one or more steps or operations for configuring submetric targets based on the submetric categories and the demographic breakdown of the patient population (Step 514). In accordance with certain aspects of the present disclosure, routine 500 may comprise one or more steps or operations for configuring an equitable benefit framework comprising the submetric categories and weights and the submetric targets (Step 516). In accordance with certain aspects of the present disclosure, routine 500 may comprise one or more steps or operations for storing the EB framework in a memory device for at least one server or computing device (Step 518).

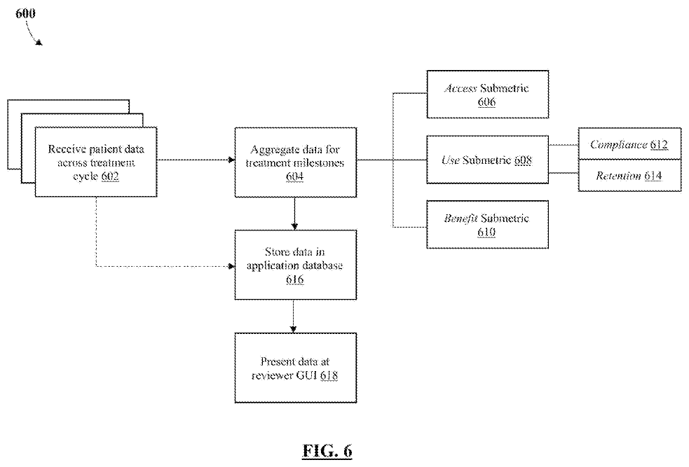

Referring now to FIG. 6, a process flow diagram of a routine 600 for collecting submetric data across a treatment cycle for a digital product or service is shown. In accordance with certain aspects of the present disclosure, routine 600 may be embodied within one or more system routine of system 100, as shown in FIG. 1, and/or system 200, as shown in FIG. 2. The digital product or service may comprise a DHI or an SaMD platform and/or product and may be configured as a therapeutic or diagnostic tool for treating or diagnosing a targeted medical condition (e.g., pediatric ADHD). In accordance with certain aspects of the present disclosure, routine 600 may be successive or sequential to routine 500 of FIG. 5 and/or may comprise one or more subroutine of routine 500 of FIG. 5. In accordance with certain aspects of the present disclosure, routine 600 may comprise one or more steps or operations for receiving patient activity data across a treatment cycle for the digital product or service (Step 602). In accordance with certain aspects of the present disclosure, routine 600 may comprise one or more steps or operations for aggregating patient activity for treatment milestones across the treatment cycle for the digital product or service (Step 604). In accordance with certain aspects of the present disclosure, the treatment milestones may comprise one or more submetrics. In accordance with certain embodiments, the one or more submetrics may comprise an Access submetric 606, a Use submetric 608, and a Benefit submetric 610. In accordance with certain embodiments, Use submetric 608 may comprise a Compliance submetric 612 and a Retention submetric 614. In accordance with certain aspects of the present disclosure, routine 600 may comprise one or more steps or operations for storing the patient activity data and the submetric data in the application database (Step 616) and presenting the data at a graphical user interface of a review application (Step 618).

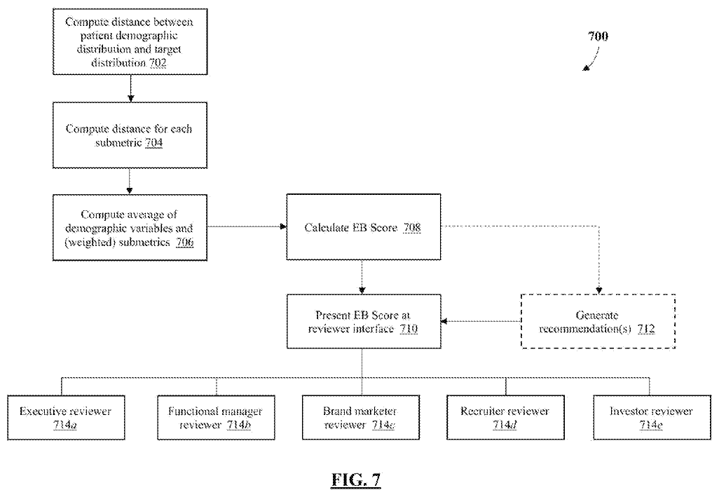

Referring now to FIG. 7, a process flow diagram of a routine 700 for calculating an equitable benefit score for a digital product or service is shown. In accordance with certain aspects of the present disclosure, routine 700 may be embodied within one or more system routine of system 100, as shown in FIG. 1, and/or system 200, as shown in FIG. 2. The digital product or service may comprise a DHI or an SaMD platform and/or product and may be configured as a therapeutic or diagnostic tool for treating or diagnosing a targeted medical condition (e.g., pediatric ADHD). In accordance with certain aspects of the present disclosure, routine 700 may be successive or sequential to routine 500 of FIG. 5 and/or routine 600 of FIG. 6 and/or may comprise one or more subroutine of routine 500 of FIG. 5 and/or subroutine 600 of FIG. 6. In accordance with certain aspects of the present disclosure, routine 700 may comprise one or more steps or operations for computing a distance between a patient demographic distribution for the digital product or service and a target distribution for the targeted medical condition (Step 702). In accordance with certain aspects of the present disclosure, routine 700 may comprise one or more steps or operations for computing a distance for each submetric in the equitable benefit framework and a target for each submetric (Step 704). In accordance with certain aspects of the present disclosure, routine 700 may comprise one or more steps or operations for computing an average of all demographic variables and weighted submetrics (Step 706). In accordance with certain aspects of the present disclosure, routine 700 may comprise one or more steps or operations for calculating an equitable benefit score for the digital product or service (e.g., DHI/SaMD) (Step 708) and present the EB score at a graphical user interface of a reviewer application (Step 710). In accordance with certain embodiments, routine 700 may comprise one or more steps or operations for generating one or more recommendation(s) for improving the EB score and presenting the one or more recommendation(s) at the graphical user interface of a reviewer application (Step 712). In accordance with certain embodiments, the review application may comprise one or more user roles, including an executive reviewer 714a; a functional manager reviewer 714b; a brand marketer reviewer 714c; a recruiter reviewer 714d; and an investor reviewer 714e.

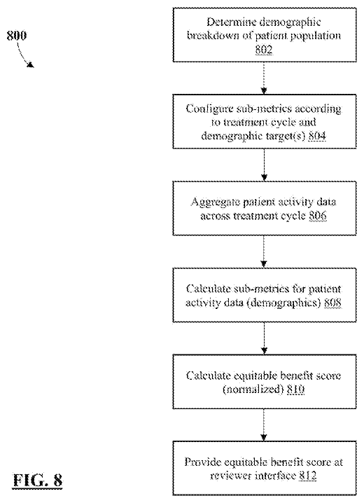

Referring now to FIG. 8, a process flow diagram of a method 800 for calculating an equitable benefit score for a digital product or service is shown. Method 800 may be embodied within one or more system operations or routines of system 100 of FIG. 1 and/or system 200 of FIG. 2. In accordance with certain aspects of the present disclosure, the digital product or service may comprise a DHI or an SaMD platform and/or product. In accordance with certain aspects of the present disclosure, the digital product or service may be configured as a digital therapy or diagnostic (e.g., DHI/SaMD) for treating or diagnosing a targeted medical condition (e.g., pediatric ADHD). In accordance with certain aspects of the present disclosure, method 800 may comprise one or more steps or operations for determining, with at least one processor, a demographic breakdown of a patient population for at least one medical condition (e.g., pediatric ADHD in America) (Step 802). Method 800 may further comprise one or more steps or operations for determining, with at least one processor communicably engaged with at least one remote server, a target demographic breakdown of a patient population for the digital product or service, wherein the digital product or service is configured as a diagnostic or therapeutic for the at least one medical condition. In accordance with certain aspects of the present disclosure, method 800 may proceed by performing one or more steps or operations for configuring, with the at least one processor, one or more benefit submetrics for the digital product or service (Step 804). In accordance with certain aspects of the present disclosure, method 800 may proceed by performing one or more steps or operations for collecting, with an application server communicably engaged with a plurality of patient devices and the at least one processor, a plurality of patient activity data for the digital product or service (Step 806). In accordance with certain aspects of the present disclosure, method 800 may proceed by performing one or more steps or operations for aggregating, with the at least one processor, the patient activity data (Step 806). In accordance with certain aspects of the present disclosure, method 800 may proceed by performing one or more steps or operations for calculating, with the at least one processor, the one or more benefit submetrics for the digital product or service according to the patient activity data (Step 808). In accordance with certain aspects of the present disclosure, method 800 may proceed by performing one or more steps or operations for processing, with the at least one processor, the one or more benefit submetrics according to an equitable benefit framework to calculate an equitable benefit score for the digital product or service (Step 810). In accordance with certain aspects of the present disclosure, method 800 may proceed by performing one or more steps or operations for providing, with the at least one processor communicably engaged with a client device, the equitable benefit score to a reviewer user at an interface of a reviewer application (Step 812).

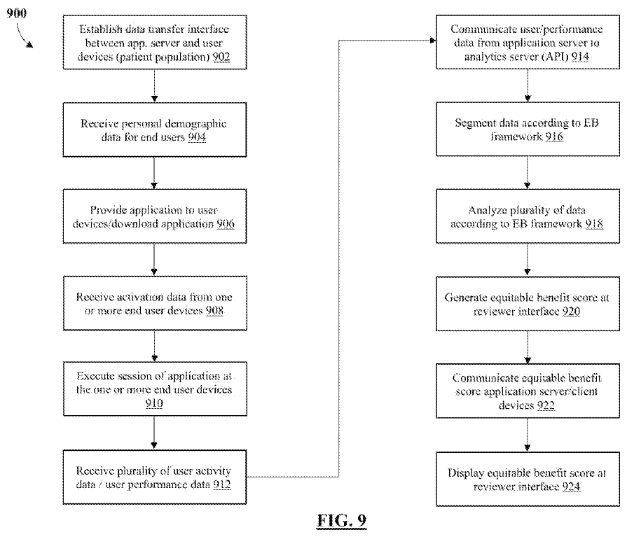

Referring now to FIG. 9, a process flow diagram of a method 900 for calculating an equitable benefit score for a digital product or service is shown. Method 900 may be embodied within one or more system operations or routines of system 100 of FIG. 1 and/or system 200 of FIG. 2. In accordance with certain aspects of the present disclosure, method 900 may comprise one or more steps or operations for establishing (e.g., via a network communications interface) a data transfer interface between an application server and a plurality of end user devices associated with a plurality of end users comprising a patient population (Step 902). Method 900 may proceed upon executing one or more steps or operations for receiving (e.g., with the application server via the data transfer interface) personal demographic data for each end user in the plurality of end users (Step 904). Method 900 may proceed upon executing one or more steps or operations for providing (e.g., with the application server) a software application to the plurality of end user devices (Step 906). Method 900 may proceed upon executing one or more steps or operations for receiving (e.g., with the application server via the data transfer interface) device activation data for the software application from one or more end user device in the plurality of end user devices (Step 908). Method 900 may proceed upon executing one or more steps or operations for executing, at one or more timepoints, one or more instances of the software application at the one or more end user device in the plurality of end user devices (Step 910). Method 900 may proceed upon executing one or more steps or operations for receiving at the one or more timepoints (e.g., with the application server via the data transfer interface) a plurality of user activity data and/or user performance data from the plurality of end user devices (Step 912). In certain embodiments, the plurality of user activity data and/or user performance data comprises session data comprising one or more user-generated inputs from one or more instances of the software application. Method 900 may proceed upon executing one or more steps or operations for communicating (e.g., with the application server) the personal demographic data for each end user in the plurality of end users and the plurality of user activity data and/or user performance data from the plurality of end user devices to an analytics server via an application programming interface (Step 914).

Method 900 may proceed upon executing one or more steps or operations for segmenting (e.g., with the analytics server) the plurality of user activity data and/or user performance data for each end user in the plurality of end users according to the personal demographic data for each end user (Step 916). Method 900 may proceed upon executing one or more steps or operations for analyzing (e.g., with the analytics server) the plurality of user activity data and/or user performance data according to an equitable benefit framework (Step 918). In certain embodiments, the equitable benefit framework comprises one or more steps or operations for calculating one or more equitable benefit submetrics for one or more subgroups of the patient population. In certain embodiments, the one or more equitable benefit submetrics comprise one or more of a user access metric, a usage metric, and a user benefit metric for each end user in the plurality of end users. Method 900 may proceed upon executing one or more steps or operations for generating (e.g., with the analytics server) an equitable benefit score for the software application (Step 920). Method 900 may proceed upon executing one or more steps or operations for communicating (e.g., with the analytics server) the equitable benefit score for the software application to a client device communicably engaged with the analytics server (Step 922) and displaying (e.g., with the client device) the equitable benefit score for the software application to at least one reviewer user via a reviewer user interface (Step 924).

In accordance with certain aspects of the present disclosure, method 900 may be further configured wherein calculating the device activation metric comprises one or more steps or operations for calculating (e.g., with the analytics server communicably engaged with at least one database) a ratio of delivered device activation codes to activated device activation codes stored in the at least one database. Method 900 may be further configured wherein calculating the user compliance metric comprises comparing (e.g., with the analytics server communicably engaged with at least one database) an average number of sessions of the software application executed by each end user in the plurality of end users within a specified time period to a target number of sessions of the software application for the specified time period. Method 900 may be further configured wherein calculating the user retention metric comprises comparing (e.g., with the analytics server communicably engaged with at least one database) a number of active sessions of the software application for each end user in the plurality of end users within a specified time period to a target number of active sessions of the software application for the specified time period. Method 900 may be further configured wherein calculating the user benefit metric comprises tracking (e.g., with the analytics server communicably engaged with at least one database) at least one globally unique identifier for each end user in the plurality of end users. The at least one globally unique identifier may be embedded in at least one uniform resource locator associated with the software application (e.g., a webpage, download link, user signup workflow, etc.). In accordance with certain embodiments, method 900 may be configured wherein receiving the personal demographic data comprises receiving (e.g., with the application server via the data transfer interface) a plurality of user-generated responses via at least one end user workflow for the software application. Method 900 may further comprise one or more steps or operations for establishing (e.g., with the analytics server or the application server) a data transfer interface with at least one electronic health records server, wherein the personal demographic data for each end user in the plurality of end users is received from the at least one electronic health records server. Method 900 may further comprise one or more steps or operations for imputing (e.g., with the analytics server) one or more personal demographic datapoint for one or more end user in the plurality of end users according to one or more demographic statistics for the one or more end user in the plurality of end users. Method 900 may further comprise one or more steps or operations for generating (e.g., with the analytics server) one or more recommendations for improving the equitable benefit score for the software application, wherein the one or more recommendations comprise recommendations for modifying one or more computerized stimuli or interactions for the software application for the one or more subgroups of the patient population.

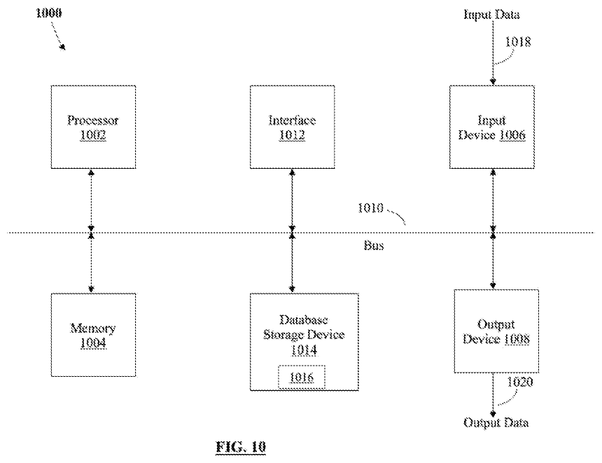

Referring now to FIG. 10, a processor-implemented computing device in which one or more aspects of the present disclosure may be implemented is shown. According to an embodiment, a processing system 1000 may generally comprise at least one processor 1002, a memory 1004, an input device 1006 for receiving input data 1018 and an output device 1008 that produces output data 1020 coupled together with at least one bus 1010. In certain embodiments, input device 1006 and output device 1008 could be the same device. An interface 1012 can also be provided for coupling the processing system 1000 to one or more peripheral devices, for example interface 1012 could be a PCI card or PC card. At least one database storage device 1014 which houses at least one database 1016 can also be provided. The memory 1004 can be any form of memory device, for example, volatile or non-volatile memory, solid state storage devices, magnetic devices, etc. The processor 1002 could comprise more than one distinct processing device, for example to handle different functions within the processing system 1000. Input device 1006 receives input data 1018 and can comprise, for example, a keyboard, a pointer device such as a pen-like device or a mouse, an audio receiving device for voice-controlled activation such as a microphone, data receiver or antenna such as a modem or wireless data adaptor, data acquisition card, etc. Input data 1018 could come from different sources, for example keyboard instructions in conjunction with data received via a network. Output device 1008 produces or generates output data 1020 and can comprise, for example, a display device or monitor in which case output data 1020 is visual, a printer in which case output data 1020 is printed, a port for example a USB port, a peripheral component adaptor, a data transmitter or antenna such as a modem or wireless network adaptor, etc. Output data 1020 could be distinct and derived from different output devices, for example a visual display on a monitor in conjunction with data transmitted to a network. A user could view data output, or an interpretation of the data output, on, for example, a monitor or using a printer. The storage device 1014 can be any form of data or information storage means, for example, volatile or non-volatile memory, solid state storage devices, magnetic devices, and the like.

In use, the processing system 1000 is adapted to allow data or information to be stored in and/or retrieved from, via wired or wireless communication means, at least one database 1016. The interface 1012 may allow wired and/or wireless communication between the processing unit 1002 and peripheral components that may serve a specialized purpose. In general, the processor 1002 can receive instructions as input data 1018 via input device 1006 and can display processed results or other output to a user by utilizing output device 1008. More than one input device 1006 and/or output device 1008 can be provided. It should be appreciated that the processing system 1000 may be any form of terminal, server, specialized hardware, or the like.

It is to be appreciated that the processing system 1000 may be a part of a networked communications system. Processing system 1000 could connect to a network, for example the Internet or a WAN. Input data 1018 and output data 1020 could be communicated to other devices via the network. The transfer of information and/or data over the network can be achieved using wired communications means or wireless communications means. A server can facilitate the transfer of data between the network and one or more databases. A server and one or more databases provide an example of an information source.

Thus, the processing computing system environment 1000 illustrated in FIG. 10 may operate in a networked environment using logical connections to one or more remote computers. The remote computer may be a personal computer, a server, a router, a network PC, a peer device, or other common network node, and typically includes many or all of the elements described above.

It is to be further appreciated that the logical connections depicted in FIG. 10 include a local area network (LAN) and a wide area network (WAN) but may also include other networks such as a personal area network (PAN). Such networking environments are commonplace in offices, enterprise-wide computer networks, intranets, and the Internet. For instance, when used in a LAN networking environment, the computing system environment 1000 is connected to the LAN through a network interface or adapter. When used in a WAN networking environment, the computing system environment typically includes a modem or other means for establishing communications over the WAN, such as the Internet. The modem, which may be internal or external, may be connected to a system bus via a user input interface, or via another appropriate mechanism. In a networked environment, program modules depicted relative to the computing system environment 1000, or portions thereof, may be stored in a remote memory storage device. It is to be appreciated that the illustrated network connections of FIG. 10 are exemplary and other means of establishing a communications link between multiple computers may be used.

FIG. 10 is intended to provide a brief, general description of an illustrative and/or suitable exemplary environment in which various embodiments of the invention may be implemented. FIG. 10 is an example of a suitable environment and is not intended to suggest any limitation as to the structure, scope of use, or functionality of an embodiment of the present invention. A particular environment should not be interpreted as having any dependency or requirement relating to any one or combination of components illustrated in an exemplary operating environment. For example, in certain instances, one or more elements of an environment may be deemed not necessary and omitted. In other instances, one or more other elements may be deemed necessary and added.

In the preceding description, certain embodiments may be described with reference to acts and symbolic representations of operations that are performed by one or more computing devices, such as the computing system 1000 of FIG. 10. As such, it will be understood that such acts and operations, which are at times referred to as being computer-executed, include the manipulation by the processor of the computer of electrical signals representing data in a structured form. This manipulation transforms the data or maintains them at locations in the memory system of the computer, which reconfigures or otherwise alters the operation of the computer in a manner understood by those skilled in the art. The data structures in which data is maintained are physical locations of the memory that have particular properties defined by the format of the data. However, while an embodiment is being described in the foregoing context, it is not meant to be limiting as those of skill in the art will appreciate that the acts and operations described hereinafter may also be implemented in hardware.

Embodiments of the present invention can be implemented with numerous other general-purpose or special-purpose computing devices, systems or configurations. Examples of well-known computing systems, environments, and configurations suitable for use in embodiment of the invention include, personal computers, handheld or laptop devices, personal digital assistants, multiprocessor systems, microprocessor-based systems, set top boxes, programmable consumer electronics, network, minicomputers, server computers, game server computers, web server computers, mainframe computers, and distributed computing environments that include any of the above systems or devices.

Various embodiments of the invention will be described herein in a general context of computer-executable instructions, such as program modules, being executed by a computer. Generally, program modules include routines, programs, objects, components, data structures, etc., that perform particular tasks or implement particular abstract data types. In certain embodiments, distributed computing environments where tasks are performed by remote processing devices that are linked through a communications network may also be employed. In distributed computing environments, program modules may be located in both local and remote computer storage media including memory storage devices.

As will be appreciated by one of skill in the art, the present invention may be embodied as a method (including, for example, a computer-implemented process, a business process, and/or any other process), apparatus (including, for example, a system, machine, device, computer program product, and/or the like), or a combination of the foregoing. Accordingly, embodiments of the present invention may take the form of an entirely hardware embodiment, an entirely software embodiment (including firmware, resident software, micro-code, etc.), or an embodiment combining software and hardware aspects that may generally be referred to herein as a “system.” Furthermore, embodiments of the present invention may take the form of a computer program product on a computer-readable medium having computer-executable program code embodied in the medium.

Any suitable transitory or non-transitory computer readable medium may be utilized. The computer readable medium may be, for example but not limited to, an electronic, magnetic, optical, electromagnetic, infrared, or semiconductor system, apparatus, or device. More specific examples of the computer readable medium include, but are not limited to, the following: an electrical connection having one or more wires; a tangible storage medium such as a portable computer diskette, a hard disk, a random access memory (RAM), a read-only memory (ROM), an erasable programmable read-only memory (EPROM or Flash memory), a compact disc read-only memory (CD-ROM), or other optical or magnetic storage device.

In the context of this document, a computer readable medium may be any medium that can contain, store, communicate, or transport the program for use by or in connection with the instruction execution system, apparatus, or device. The computer usable program code may be transmitted using any appropriate medium, including but not limited to the Internet, wireline, optical fiber cable, radio frequency (RF) signals, or other mediums.

Computer-executable program code for carrying out operations of embodiments of the present invention may be written in an object oriented, scripted or unscripted programming language such as Java, Perl, Smalltalk, C++, or the like. However, the computer program code for carrying out operations of embodiments of the present invention may also be written in conventional procedural programming languages, such as the “C” programming language or similar programming languages.

Embodiments of the present invention are described above with reference to flowchart illustrations and/or block diagrams of methods, apparatus (systems), and computer program products. It will be understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general-purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks.

These computer-executable program code portions (i.e., computer-executable instructions) may also be stored in a computer-readable memory that can direct a computer or other programmable data processing apparatus to function in a particular manner, such that the code portions stored in the computer readable memory produce an article of manufacture including instruction mechanisms which implement the function/act specified in the flowchart and/or block diagram block(s). Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

The computer-executable program code may also be loaded onto a computer or other programmable data processing apparatus to cause a series of operational phases to be performed on the computer or other programmable apparatus to produce a computer-implemented process such that the code portions which execute on the computer or other programmable apparatus provide phases for implementing the functions/acts specified in the flowchart and/or block diagram block(s). Alternatively, computer program implemented phases or acts may be combined with operator or human implemented phases or acts in order to carry out an embodiment of the invention.

As the phrases are used herein, a processor may be “operable to” or “configured to” perform a certain function in a variety of ways, including, for example, by having one or more general-purpose circuits perform the function by executing particular computer-executable program code embodied in computer-readable medium, and/or by having one or more application-specific circuits perform the function.

The terms “program” or “software” are used herein in a generic sense to refer to any type of computer code or set of computer-executable instructions that can be employed to program a computer or other processor to implement various aspects of the present technology as discussed above. Additionally, it should be appreciated that according to one aspect of this embodiment, one or more computer programs that when executed perform methods of the present technology need not reside on a single computer or processor but may be distributed in a modular fashion among a number of different computers or processors to implement various aspects of the present technology.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” As used herein, the terms “right,” “left,” “top,” “bottom,” “upper,” “lower,” “inner” and “outer” designate directions in the drawings to which reference is made.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The present disclosure includes that contained in the appended claims as well as that of the foregoing description. Although this invention has been described in its exemplary forms with a certain degree of particularity, it is understood that the present disclosure of has been made only by way of example and numerous changes in the details of construction and combination and arrangement of parts may be employed without departing from the spirit and scope of the invention.

What is claimed is:

1. A method comprising:
establishing, via a network communications interface, a data transfer interface between an application server and a plurality of end user devices associated with a plurality of end users comprising a patient population;
receiving, with the application server via the data transfer interface, personal demographic data for each end user in the plurality of end users;
providing, with the application server, a software application to the plurality of end user devices, wherein the software application comprises a diagnostic or therapeutic application for at least one medical condition associated with the patient population;
receiving, with the application server via the data transfer interface, device activation data for the software application from one or more end user device in the plurality of end user devices;
executing, at one or more timepoints, one or more instances of the software application at the one or more end user device in the plurality of end user devices;
receiving, at the one or more timepoints with the application server via the data transfer interface, a plurality of user activity data from the plurality of end user devices, wherein the plurality of user activity data comprises session data and one or more user-generated inputs from the one or more instances of the software application;
communicating, with the application server, the personal demographic data for each end user in the plurality of end users and the plurality of user activity data from the plurality of end user devices to an analytics server via an application programming interface;
segmenting, with the analytics server, the plurality of user activity data for each end user in the plurality of end users according to the personal demographic data for each end user;
analyzing, with the analytics server, the plurality of user activity data according to an equitable benefit framework,
wherein the equitable benefit framework comprises calculating one or more equitable benefit submetrics for one or more subgroups of the patient population,
wherein the one or more equitable benefit submetrics comprise one or more of an access metric, a usage metric, and a user benefit metric for each end user in the plurality of end users;
generating, with the analytics server, an equitable benefit score for the software application, wherein the equitable benefit score comprises comparing one or more calculated equitable benefit submetrics for the one or more subgroups of the patient population to one or more targeted distribution metrics for the patient population;
communicating, with the analytics server, the equitable benefit score for the software application to a client device communicably engaged with the analytics server; and
displaying, with the client device, the equitable benefit score for the software application to at least one business user.

2. The method of claim 1 wherein calculating the access metric comprises calculating, with the analytics server communicably engaged with at least one database, a ratio of delivered device activation codes to activated device activation codes stored in the at least one database.

3. The method of claim 1 wherein calculating the usage metric comprises comparing, with the analytics server communicably engaged with at least one database, an average number of sessions of the software application executed by each end user in the plurality of end users within a specified time period to a target number of sessions of the software application for the specified time period.

4. The method of claim 1 wherein calculating the usage metric comprises comparing, with the analytics server communicably engaged with at least one database, a number of active sessions of the software application for each end user in the plurality of end users within a specified time period to a target number of active sessions of the software application for the specified time period.

5. The method of claim 1 wherein calculating the user benefit metric comprises tracking, with the analytics server communicably engaged with at least one database, at least one globally unique identifier for each end user in the plurality of end users, wherein the at least one globally unique identifier is embedded in at least one uniform resource locator associated with the software application.

6. The method of claim 1 wherein receiving the personal demographic data comprises receiving, with the application server via the data transfer interface, a plurality of user-generated responses via at least one end user workflow for the software application.

7. The method of claim 1 further comprising establishing, with the analytics server or the application server, a data transfer interface with at least one electronic health records server, wherein the personal demographic data for each end user in the plurality of end users is received from the at least one electronic health records server.

8. The method of claim 1 further comprising imputing, with the analytics server, one or more personal demographic datapoint for one or more end user in the plurality of end users according to one or more demographic statistics for the one or more end user in the plurality of end users.

9. The method of claim 1 further comprising generating, with the analytics server, one or more recommendations for improving the equitable benefit score for the software application, wherein the one or more recommendations comprise recommendations for modifying one or more computerized stimuli or interactions for the software application for the one or more subgroups of the patient population.

10. A system comprising:
an application server comprising a software application hosted thereon, the software application comprising a diagnostic or therapeutic application for at least one medical condition associated with a patient population;
a plurality of end user computing devices communicably engaged with the application server via a network communications interface, the plurality of end user computing devices being associated with a plurality of end users comprising the patient population;
an analytics server communicably engaged with the application server via at least one application programming interface; and
a business user computing device communicably engaged with the analytics server,
wherein the application server is configured to execute one or more operations comprising:
receiving personal demographic data for each end user in the plurality of end users from the plurality of end user computing devices,
providing an instance of the software application to the plurality of end user computing devices,
receiving device activation data for the software application from one or more end user device in the plurality of end user computing devices,
receiving, at one or more timepoints, a plurality of user activity data from the plurality of end user computing devices, wherein the plurality of user activity data comprises session data and one or more user-generated inputs from one or more sessions of the software application, and
communicating the personal demographic data and the plurality of user activity data to the analytics server via the at least one application programming interface;
wherein the analytics server is configured to execute one or more operations comprising:
segmenting the plurality of user activity data for each end user in the plurality of end users according to the personal demographic data,
analyzing the plurality of user activity data according to an equitable benefit framework, wherein the equitable benefit framework comprises calculating one or more equitable benefit submetrics for one or more subgroups of the patient population, wherein the one or more equitable benefit submetrics comprise one or more of a user access metric, a usage metric, and a user benefit metric for each end user in the plurality of end users,
generating an equitable benefit score for the software application, wherein the equitable benefit score comprises comparing one or more calculated equitable benefit submetrics for the one or more subgroups of the patient population to one or more targeted distribution metrics for the patient population, and
communicating the equitable benefit score for the software application to the business user computing device,
wherein the business user computing device is configured to display the equitable benefit score for the software application to at least one business user.

11. The system of claim 10 wherein calculating the access metric comprises calculating a ratio of delivered device activation codes to activated device activation codes.

12. The system of claim 10 wherein calculating the usage metric comprises comparing an average number of sessions of the software application executed by each end user in the plurality of end users within a specified time period to a target number of sessions of the software application for the specified time period.

13. The system of claim 10 wherein calculating the usage metric comprises comparing a number of active sessions of the software application for each end user in the plurality of end users within a specified time period to a target number of active sessions of the software application for the specified time period.

14. The system of claim 10 further comprising at least one electronic health records server communicably engaged with the analytics server or the application server, wherein the at least one electronic health records server is configured to communicate the personal demographic data for each end user in the plurality of end users to the analytics server or the application server via a second at least one application programming interface.

15. The system of claim 10 wherein the software application comprises one or more computerized stimuli or interactions configured to prompt one or more time varying responses from each end user in the plurality of end users via the plurality of end user computing devices, wherein the one or more computerized stimuli or interactions are associated with at least one computerized task.

16. The system of claim 15 wherein the analytics server is further configured to execute one or more operations for generating one or more recommendations for improving the equitable benefit score for the software application, wherein the one or more recommendations comprise recommendations for modifying the one or more computerized stimuli or interactions for at least one end user in the plurality of end users.

17. The system of claim 16 wherein the application server is further configured to execute one or more operations for modifying the one or more computerized stimuli or interactions for the at least one end user in the plurality of end users according to the one or more recommendations for improving the equitable benefit score for the software application.

18. The system of claim 16 wherein the business user computing device is configured to display the one or more recommendations for improving the equitable benefit score for the software application to the at least one business user.

19. The system of claim 17 wherein the analytics server is further configured to execute one or more operations for determining a measure of change in the equitable benefit score for the software application in response to modifying the one or more computerized stimuli or interactions for the at least one end user in the plurality of end users.

20. A non-transitory computer-readable medium having processor-executable instructions stored thereon that, when executed, command at least one processor to perform one or more operations, the one or more operations comprising:
establishing a data transfer interface between an application server and a plurality of end user devices associated with a plurality of end users comprising a patient population;
receiving personal demographic data for each end user in the plurality of end users;
providing a software application to the plurality of end user devices, wherein the software application comprises a diagnostic or therapeutic application for at least one medical condition associated with the patient population;
receiving device activation data for the software application from one or more end user device in the plurality of end user devices;
receiving a plurality of user activity data from the plurality of end user devices, wherein the plurality of user activity data comprises session data and one or more user-generated inputs from one or more instances of the software application;
segmenting the plurality of user activity data for each end user in the plurality of end users according to the personal demographic data for each end user;
analyzing the plurality of user activity data according to an equitable benefit framework,
wherein the equitable benefit framework comprises calculating one or more equitable benefit submetrics for one or more subgroups of the patient population,
wherein the one or more equitable benefit submetrics comprise one or more of an access metric, a usage metric, and a user benefit metric for each end user in the plurality of end users;
generating an equitable benefit score for the software application, wherein the equitable benefit score comprises comparing one or more calculated equitable benefit submetrics for the one or more subgroups of the patient population to one or more targeted distribution metrics for the patient population; and
communicating the equitable benefit score for the software application to a client device.
